(19) United States
(12) Patent Application Publication
(10) Pub. No.: US 2015/0099972 A1
(43) Pub. Date: Apr. 9, 2015

(54) MYOGRAPHY METHOD AND SYSTEM

(71) Applicant: Vibrado Technologies, Inc., Sunnyvale, CA (US)

(72) Inventors: Quinn A. Jacobson, Sunnyvale, CA (US); Cynthia Kuo, Mountain View, CA (US)

(21) Appl. No.: 14/506,322

(22) Filed: Oct. 3, 2014

Related U.S. Application Data
(60) Provisional application No. 61/886,782, filed on Oct. 4, 2013.

(51) Int. Cl. A61B 5/00; A61B 5/0488; A61B 8/08

(52) U.S. Cl. CPC A61B 5/4519; A61B 8/08; A61B 5/0082; A61B 5/0488
USPC 600/438; 600/595; 600/407; 600/473; 600/476; 600/546

(57) ABSTRACT

A myography system and method involves the use of reflected energy and/or pass-through energy to determine muscle activity as an alternative or in addition to using native signals which originate from the muscle being studied.

### CROSS-REFERENCE TO RELATED APPLICATION

This application claims the benefit of U.S. Provisional Application No. 61/886,782, filed Oct. 4, 2013, which is incorporated herein by reference in its entirety and for all purposes.

### FIELD OF THE INVENTION

The invention relates, in general, to signal processing and more particularly to analysis of data related to muscle activity.

### BACKGROUND OF THE INVENTION

The study of muscular contraction is called myography. Myography has many applications such as physical therapy and sports training Myography allows a therapist or trainer to observe the muscle activity and fatigue of a person's muscles. Information obtained from the observations can be used to access progress in strengthening the muscles or in the efficiency of using muscles.

A muscle contraction occurs when the central nervous system sends signals to a motor neuron in the spinal cord, and the motor neuron is activated. The motor neuron releases acetylcholine, a neurotransmitter that triggers a response from a muscle fiber. Muscular contractions can be detected by measuring electrical, vibrational, or audio signals indicative of muscle activity. See, for example, U.S. Patent Application Publication Nos. 2010/0262042 (entitled “Acoustic Myography Systems and Methods”), 2010/0268080 (entitled “Apparatus and Technique to Inspect Muscle Function”), 2012/0157886 (entitled “Mechanomyography Signal Input Device, Human-Machine Operating System and Identification Method Thereof”), 2012/0188158 (entitled “Wearable Electromyography-based Human-Computer Interface), 2013/0072811 (entitled “Neural Monitoring System”), and 2013/0289434 (entitled “Device for Measuring and Analyzing Electromyography Signals”).

Electromyography measures electrical impulses from muscles. Acoustic myography detects low frequency sounds, which are mostly inaudible, created during muscle activity. Mechanomyography detects small vibrations of the muscle when it contracts. The vibrations, which can be in the range of a few vibrations per second to a few hundred vibrations per section, are virtually invisible to the naked eye.

Myography is typically confined to detecting energy signals that originate from muscles. Contraction of a muscle fiber generates an electric field, and the potential can be measured directly by electrodes in the muscle fiber or indirectly by electrodes on the surface of the skin. The electric field potential (electrical signals) used in electromyography originates from the muscle. The low frequency sounds (audio signals) used in acoustic myography and the vibrations (vibrational signals) used in mechanomyography also originate from the muscle.

In order to detect these signals which originate from the muscle, conventional myography techniques commonly rely on devices attached firmly to and/or placed in contact with the body. For example, electromyography typically uses electrodes in the muscle or placed in contact with the skin. Mechanomyography typically uses sensors (such as piezoelectric sensors and miniature accelerometers) firmly attached to the body or in contact with skin directly above the muscle being studied. Acoustic myography uses microphones placed as close as possible to the muscle to minimize background noise. A gel is often used to mount the microphone on the skin surface to enhance sound transfer to the microphone as discussed by Harrison et al., “Portable acoustic myography—a realistic noninvasive method for assessment of muscle activity and coordination in human subjects in most home and sports settings,” Physiol Rep. vol. 1(2), pp 1-9 (2013).

One can also observe muscles by performing ultrasound imaging. This is described by P W Hodges et al. in “Measurement of muscle contraction with ultrasound imaging,” Muscle Nerve. 2003 June; 27(6):682-92. Like the above myography approaches this requires an ultrasound imaging sensor to be in direct pressure contact with the skin above the muscle being observed.

The need for firm attachment and/or contact between sensors and the person often limits myography to a laboratory setting or other highly controlled conditions. In some situations, firm attachment and/or contact between sensors and the person may not be possible or feasible. Also, even with firm attachment and/or contact, the presence of fat between the skin surface and the muscle may hamper detection of electrical, audio, and vibration signals that originate from the muscle.

Accordingly, there is a need for a myography system and method that do not rely on a sensor to be firmly attached to or in contact with the body. What is also needed are a myography system and method that can utilize artifacts related to muscle activity other than the electrical, audio, and vibrational energy originating from the muscle itself.

### SUMMARY

Briefly and in general terms, the present invention is directed to a myography method, a myography system, and a non-transitory computer readable medium for performing myography.

In aspects of the invention, a myography method comprises transmitting energy toward a body having a muscle, detecting resultant energy from the body, and determining muscle activity of the muscle using at least the resultant energy that was detected.

In aspects of the invention, a myography system comprises a transmitter configured to transmit energy toward a body having a muscle, a receiver configured to detect resultant energy from the body, and a processor configured to determine muscle activity of the muscle using at least the resultant energy that was detected.

In aspects of the invention, a non-transitory computer readable medium has a stored computer program embodying instructions, which when executed by a computer, causes the computer to perform myography, and the computer readable medium comprises instructions for transmitting energy toward a body having a muscle, instructions for detecting resultant energy from the body, and instructions for determining muscle activity of the muscle using at least the resultant energy that was detected.

In the above aspects, the resultant energy can be any one or both of reflected energy and pass-through energy. The reflected energy includes at least some of the transmitted energy that was reflected by the body. The pass-through energy includes at least some of the transmitted energy that traversed across the body.

The features and advantages of the invention will be more readily understood from the following detailed description which should be read in conjunction with the accompanying drawings.

### BRIEF DESCRIPTION OF THE DRAWINGS

The drawings are not necessarily to scale.

FIG. 1 is a schematic sectional diagram of a part of human body, the diagram showing a sensor for detecting energy reflected from the body.

FIG. 2 is a schematic diagram showing a system for determining muscle activity from energy reflected from the body and/or energy passed through the body.

FIGS. 3A and 3B are graphs showing exemplary muscle vibration data derived from energy reflected from the body and/or energy passed through the body

FIG. 4 is a flow diagram showing a myography method.

FIG. 5 is a schematic sectional diagram of a part of human body, the diagram showing a sensor for detecting energy having passed through the body.

FIG. 6 is a flow diagram showing a myography method.

FIG. 7 is a schematic sectional diagram of a part of human body, the diagram showing a sensor for detecting energy reflected from the body and energy having passed through the body.

FIGS. 8 and 9 are schematic diagrams showing systems for determining muscle activity from energy reflected from the body and/or energy passed through the body.

### INCORPORATION BY REFERENCE

All publications and patent applications mentioned in the present specification are herein incorporated by reference to the same extent as if each individual publication or patent application was specifically and individually indicated to be incorporated by reference. To the extent there are any inconsistent usages of words and/or phrases between an incorporated publication or patent and the present specification, these words and/or phrases will have a meaning that is consistent with the manner in which they are used in the present specification.

### DETAILED DESCRIPTION

While some exemplary embodiments are described in the context of a person, the method and means for analysis of the present invention can apply equally to human and animal bodies. As used herein the term “body” refers to any type of body that can be subjected to myography unless specified otherwise.

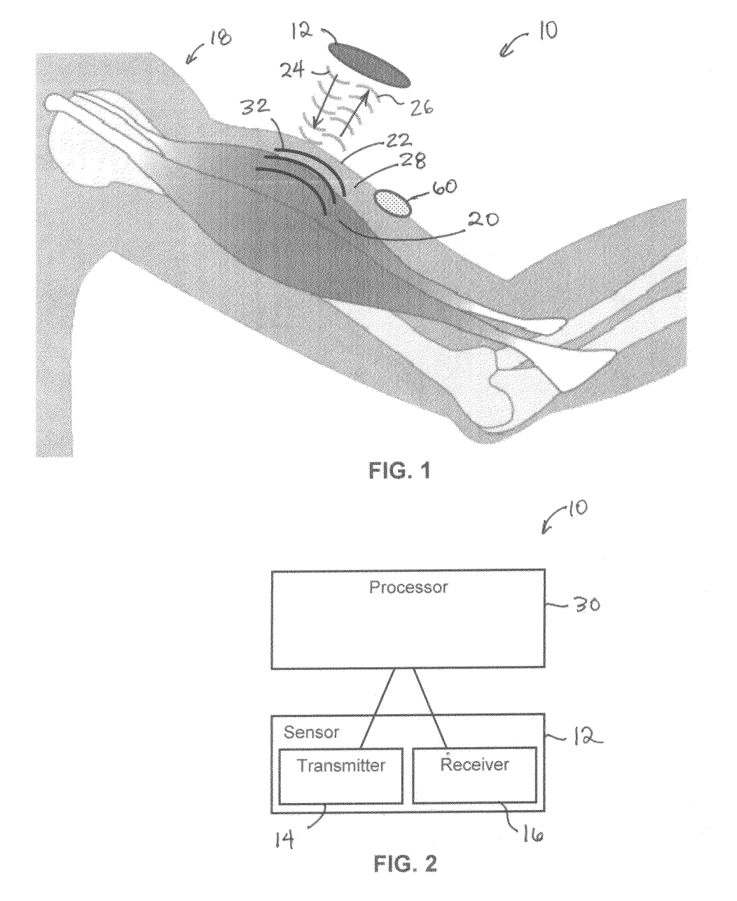

Referring now in more detail to the exemplary drawings for purposes of illustrating exemplary aspects of the invention, wherein like reference numerals designate corresponding or like elements among the several views, there is shown in FIGS. 1 and 2 myography system 10 including sensor 12. Sensor 12 includes transmitter 14 and receiver 16.

Sensor 12 is shown next to body 18 and is aimed toward muscle 20 below skin surface 22. Transmitter 14 is configured to transmit energy 24 toward body 18. Receiver 16 is configured to detect energy 26 reflected from body 18. Reflected energy 26 includes at least some of transmitted energy 24 which was reflected by body 18. Reflected energy 26 can be a reflection of transmitted energy 24 from skin surface 22 and/or from subcutaneous tissue. Subcutaneous tissue includes any of muscle 20, fat 28, or any other tissue below skin surface 22.

In the figures herein, only a portion of body 18 is illustrated. In particular, a human arm having a biceps muscle is illustrated. Although various descriptions herein may refer to an illustration of the human arm, it will be appreciated that system and method of the present invention can be applied to any muscle. Other muscles include without limitation calf, quadriceps, and hamstring muscles in addition to hand, foot, neck, back, and facial muscles.

Processor 30 is communicatively coupled to sensor 12 by any means including without limitation an electrical wire connection, optic fiber connection, and wireless communication. Processor 30 provides control signals for operating transmitter 14 and receives data from receiver 16. The data are representative of reflected energy 26. Processor 30 can include one or more electronic semiconductor chips and/or signal processing circuitry. Processor 30 may also include one or more memory devices for volatile and/or non-volatile data storage.

System 10 uses reflected energy 26 to determine activity of muscle 20. Muscle activity is associated with vibrations generated by muscle 20. The vibrations cause skin surface 22 and/or subcutaneous tissue to change position relative to sensor 12. In particular, the vibrations cause skin surface 22 and/or subcutaneous tissue to move closer and away from sensor 12. The movement is schematically represented by lines 32. Processor 30 analyzes reflected energy 26 to detect movement 32 and thereby determine muscle activity. Processor 30 can utilize known mechanomyography algorithms to determine muscle activity. Processor 30 can utilize known mechanomyography algorithms to predict one or more physiological states of muscle 20.

Processor 30 can convert data obtained from receiver 16 from the time domain to the frequency domain, apply heuristics, and analyze the frequency spectra to determine muscle activity as described in US Publication No. 2014/0163412 (entitled “Myography Method and System”).

Transmitted energy 24 can include sound energy, electromagnetic energy, or a combination thereof. For example, transmitter 14 can be configured to transmit (and receiver 16 can be configured to detect) audio signals, which can include acoustic waves, ultrasonic waves, or both. Acoustic waves are in the range of 20 Hz to 20 kHz and include frequencies audible to humans. Ultrasonic waves have frequencies greater than 20 kHz. Additionally or alternatively, transmitter 14 can be configured to transmit (and receiver 16 can be configured to detect) radio waves. For example, radio waves can have frequencies from 300 GHz to as low as 3 kHz. Additionally or alternatively, transmitter 14 can be configured to transmit (and receiver 16 can be configured to detect) infrared light or other frequencies of light. For example, infrared light can have frequencies in the range of 700 nm to 1 mm.

For example and without limitation, receiver 16 can be configured to detect energy in ways similar to those used in equipment for medical and security imaging. For example, known technology used for medical sonograms and ultrasound imaging can be adapted and/or modified to construct receiver 16. Unlike conventional ultrasound imaging devices, however, receiver 16 is configured to detect sound energy which has traveled through air, clothing, or other material between the skin surface and receiver 16. Receiver 16 may, but need not be, pressed against the skin surface. Also, technology used in millimeter wave scanners, such as used for full body scanning at airports, can be adapted and/or modified to construct receiver 16.

To determine muscle activity, processor 30 is configured to derive muscle vibration data from reflected energy 26 and then use the muscle vibration data to make a determination on muscle activity.

To derive vibration data, processor 30 can perform range finding analysis to measure distance between sensor 12 and body 18, and more particularly to measure distance between sensor 12 and skin surface 22 and/or subcutaneous tissue of body 18. The distance can be measured by calculating the time it takes for transmitted energy 24 to reflect off body 18 and then reach receiver 16. The measurement can be taken many times per second, such as hundreds or thousands of times per second, in order to detect vibrational movements 32 at skin surface 22 and/or subcutaneous tissue.

Additionally or alternatively, processor 30 can perform Doppler shift analysis to derive the muscle vibration data. Doppler shift analysis involves analyzing reflected energy 26 for slight variations in frequency in reflected energy 26. The slight variations, known as Doppler Effect or Doppler shift, are caused by movement of the reflective structure. Here, Doppler shift results from movement of skin surface 22 and/or subcutaneous tissue (such as muscle 20 and/or fat 28). The frequency, strength, and type (sound versus electromagnetic) of transmitted energy 24 can be selected to reflect from skin surface 22. Additionally or alternatively, the transmitted energy characteristics (frequency, strength, and type) can be selected to penetrate skin surface 22 and reflect from subcutaneous tissue. Skin penetration may be desired in cases when vibrational movement 32 cannot be reliably detected from skin surface 22.

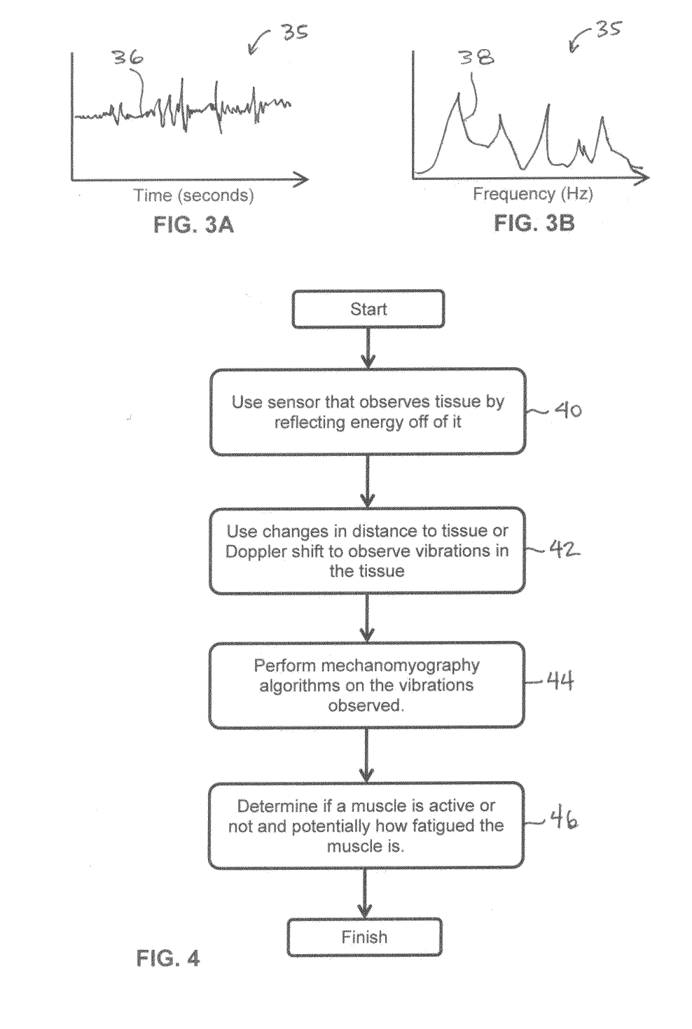

Vibration data 35 can be in the form of time domain data 36 as shown in FIG. 3A, or frequency domain data 38 as shown in FIG. 3B. Time domain data 36 show amplitudes of vibration over a period of time. Time domain data 36 can transformed to obtain frequency domain data 38. The transformation can be accomplished by well-known mathematical processes, such as Fourier transforms. Since vibration inferred from reflected energy 26 includes many different frequencies, frequency domain data 38 will show a distribution of the various frequencies and can be used to perform frequency analysis to determine which frequencies are present and/or which frequencies are dominant.

It is to be understood that the curves shown for time domain data 36 in FIG. 3A and frequency domain data 38 in FIG. 3B are simplified representations and are not intended to be an accurate representation of a particular type of muscle activity or a mathematically precise representation of time-to-frequency domain transformation.

When determining muscle activity, processor 30 can provide a prediction of one or more physiological states of muscle 20. For example, processor 30 can be configured to predict the degree to which muscle 20 is fatigued. Processor 30 can also be configured to predict whether the muscle is presently contracting or not contracting. Processor 30 can make the predictions by analyzing vibration data (36 and/or 38) to look for trends, time domain signatures, and/or frequency domain signatures associated with physiological states, including without limitation fatigue and contraction. Processor 30 can make the predictions by comparing and/or matching vibration data (36 and/or 38) against trends, time domain signatures, and/or frequency domain signatures associated with physiological states, including without limitation fatigue and contraction. The trends, time domain signatures, and/or frequency domain signatures can be part of a mechanomyography algorithm and/or stored in memory elements of processor 30 or in a database communicatively coupled to processor 30.

The transmitted energy characteristics (frequency, strength, and type) can be selected to reflect from skin surface 22 and/or subcutaneous tissue so that processor 30 can measure a change in the shape or contour of muscle 20 in addition to or as an alternative to measuring muscle vibration.

FIG. 4 depicts an exemplary myography method. Although the method will be described with reference to system 10, it will be appreciated that a system configured in other ways could be used to perform the method.

At block 40, sensor 12 is used to observe human tissue from a distance by reflecting energy off the tissue. Observation of the tissue is performed by transmitting energy 24 toward the tissue and detecting energy 26 reflected from the tissue. Transmitted energy 24 could be for example ultrasonic audio waves or other forms of energy previously mentioned.

Sensor 12 may be placed at any distance from the person. Sensor 12 can also be placed against the body of the person. For example and without limitation, sensor 12 can be embedded in furniture (e.g., chair), placed on sporting equipment (e.g., backpack chest strap, treadmill), housed in a hand-held monitoring apparatus, or mounted on a stationary structure or moving vehicle. In a moving vehicle, system 10 can be used to detect driver stress or fatigue. In these examples, sensor 12 can be out of contact with body 18 (e.g., FIG. 1) or in contact with body 18. With firm attachment and/or contact, or without contact with body 18, sensor 12 is capable of emitting transmitted energy 24 and detecting the resultant energy (e.g., reflected energy 24).

At block 42, processor 30 uses either changes in distance or Doppler shift to detect small motions in the tissue. Processor 30 interprets these small motions as vibrations.

At block 44, processor 30 applies algorithms for mechanomyography to vibrational data (36 and/or 38) from sensor 12 to determine whether muscles in the vicinity of the tissue being observed are being contracted or not. Optionally, processor 30 uses mechanomyography algorithms to identify other characteristics of the muscle such as fatigue.

At block 46, processor 30 interprets the output or results from the mechanomyography algorithms to determine the muscle activity in the vicinity of the tissue being observed by the sensor. For example and without limitation, processor 30 can make the interpretation by comparing and/or matching the results to trends, time domain signatures, and/or frequency domain signatures that have been predetermined to be associated with a physiological state, including without limitation fatigue and contraction.

In the descriptions above, reflected energy 26 can be referred to as resultant energy. The word “resultant” refers to the fact that reflected energy 26 is the result of transmission of transmitted energy 24 from transmitter 14. The result of transmission of transmitted energy 24 from transmitter 14 (i.e., the resultant energy) can be in the form of pass-through energy. Pass-through energy includes at least some of the transmitted energy that traversed across a skin surface and subcutaneous tissue of the body.

Transmitter 14 and receiver 16 can be housed together in a single sensor package, as shown in FIG. 1. Also, transmitter 14 and receiver 16 can be housed separately to allow transmitter 14 to be separated from receiver 16. This can allow the user to fine tune directional aiming of transmitter 14 and receiver 16 on an individual basis. This can also allow the user to place receiver 16 at a position that allows it to detect pass-through energy.

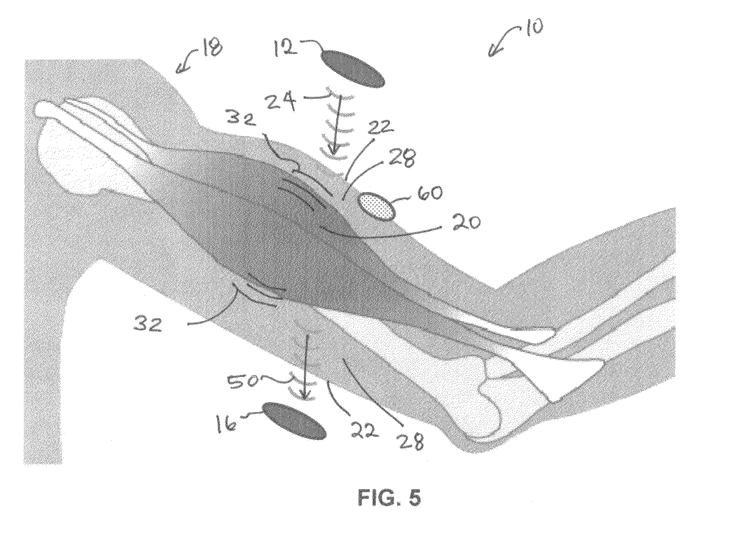

In FIG. 5, receiver 16 is placed at a position that allows it to detect pass-through energy 50 which includes at least some transmitted energy 24 that traversed across skin surface 22 and subcutaneous tissue of body 18. Subcutaneous tissue includes muscle 20 and any fat or other tissue beneath skin surface 22.

Processor 30 is configured to measure, from pass-through energy 50, a change in one or more muscle characteristics. The muscle characteristics can be energy reflectivity, energy absorption, and/or energy transmission. Processor 30 is further configured to use the measured change in the muscle characteristic(s) to determine muscle activity.

Transmitted energy 24 can be attenuated, diffused, scattered, or changed in other ways so that pass-through energy 50 differs from transmitted energy 24. Processor 30 can be configured to detect such changes and use such changes to measure an extent to which muscle 20 reflects transmitted energy 24, an extent to which muscle 20 absorbs transmitted energy 24, and/or an extent to which muscle 20 permits transmitted energy 24 to pass through (i.e., transmit through) muscle 20. Muscle 20 will reflect, absorb, and transmit energy differently depending on whether it is flexed (i.e., when it is contracted) or relaxed (i.e., when it is not contracted).

Processor 20 can be configured to use the measured change in one or more muscle characteristics (e.g., extent to which muscle 20 reflects, absorbs, and transmits energy) to predict one or more physiological states of muscle 20, including without limitation fatigue and contraction. Processor 30 can make the predictions by analyzing the measured change to look for scatter or diffusion patterns, threshold values, trends, time domain signatures, and/or frequency domain signatures associated with physiological states, including without limitation fatigue and contraction. Processor 30 can make the predictions by comparing the measured change in one or more muscle characteristics against scatter or diffusion patterns, threshold values, trends, time domain signatures, and/or frequency domain signatures associated with physiological states, including without limitation fatigue and contraction. The scatter or diffusion patterns, threshold values, trends, time domain signatures, and/or frequency domain signatures can be part of a mechanomyography algorithm and/or stored in memory elements of processor 30 or in a database communicatively coupled to processor 30.

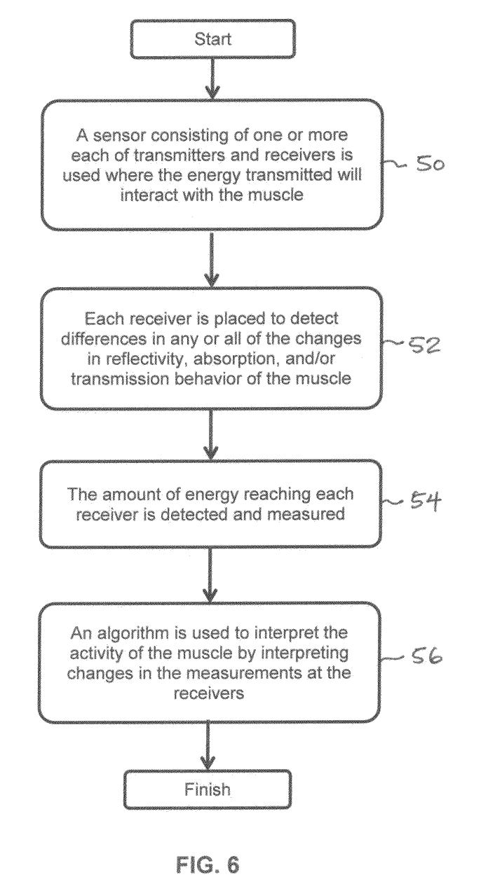

FIG. 6 depicts an exemplary myography method. Although the method will be described with reference to system 10, it will be appreciated that a system configured in other ways could be used to perform the method.

At block 50, sensor 12, which comprises transmitter 14 and receiver 16, is positioned where transmitted energy 24 will interact with muscle 20. Transmitted energy 24 could be for example ultrasonic audio waves or other forms of energy previously mentioned. Optionally, sensor 12 can comprise additional transmitters and/or additional receivers that are arranged around muscle 20. The transmitter(s) and receiver(s) may each be placed at any distance from the person, and they can also be placed against the body of the person. For example and without limitation, the transmitter(s) and receiver(s) can be embedded in furniture (e.g., chair), placed on sporting equipment (e.g., backpack chest strap, treadmill), housed in a hand-held monitoring apparatus, or mounted on a stationary structure or moving vehicle. In these examples, the transmitter(s) and receiver(s) can be out of contact with body 18 (e.g., FIG. 5) or in contact with body 18. With firm attachment and/or contact, or without contact with body 18, sensor 12 is capable of emitting transmitted energy 24 and detecting the resultant energy (e.g., pass-through 50).

At block 52, each receiver 16 is placed to detect differences in any or all of reflectivity, absorption, and/or transmission behavior of muscle 20.

At block 54, the amount of pass-through energy 50 reaching each receiver 16 is detected and measured by processor 30. For example and without limitation, interaction with muscle 20 may cause transmitted energy 24 to be attenuated, diffused, scattered, or changed in other ways so that pass-through energy 50 differs from transmitted energy 24.

At block 56, processor 30 uses an algorithm to interpret the activity of muscle 20 by interpreting changes in the measurements at each receiver 16. For example and without limitation, processor 30 can interpret the changes by comparing and/or matching the measured changes at each receiver 16 to scatter or diffusion patterns, threshold values, trends, time domain signatures, and/or frequency domain signatures that have been predetermined to be associated with a physiological state, including without limitation fatigue and contraction.

Ultrasound imaging makes use of the fact that muscles absorb or reflect a different amount sound energy depending if the muscle is relaxed or contracted. However, conventional ultrasound imaging requires sophisticated systems that produce a 2-dimensional or 3-dimensional image and that interpret the 2-dimensional or 3-dimensional image. Conclusions obtained from conventional ultrasound imaging are based on the 2-dimensional or 3-dimensional image and are not based on measurements on the level or extent of energy absorption and reflection.

In contrast, methods of the present invention need not rely on a 2-dimensional or 3-dimensional image derived from ultrasound energy. Methods of the present invention may directly leverage measurements of the energy absorption and reflection and thereby interpret the muscle state without much of the costly components of conventional ultrasound imaging. Processor 30 can be configured to measure, from data obtained from receiver 16, the level or extent to which energy is absorbed and/or reflected, and use those measurements to interpret the muscle state.

Conventional ultrasound imaging does not measure transmission of sound energy through a muscle. The transmission of sound energy through a muscle changes as the muscle contracts, with certain frequencies of sound having a much higher transmission rate through a contracted muscle versus a relaxed muscle. Processor 30 can be configured to measure, based on data obtained from receiver 16, the level or extent of transmission of sound energy through a muscle. Processor 30 can obtain measurements at selected frequencies of sound which have been predetermined to have higher transmission rates through a contracted muscle versus a relaxed muscle, and interpret any changes at the selected frequencies to make predictions on the physiological state of the muscle.

Muscles also change how much energy they absorb, reflect or transmit other frequencies of energy besides sound waves. Specific frequencies of radio waves, such as those in the MHz range (i.e., greater than 1 MHz), are reflected and transmitted at different rates by contracted muscles and and relaxed muscles. The radio waves in these frequency often have little interaction with air or typical clothing materials, so muscles can be observed with or without direct contact. Processor 30 can be configured to check certain frequencies of radio waves, for example radio waves in the MHz range, which have been predetermined to reflect and transmit at different rates by contracted muscles and relaxed muscles.

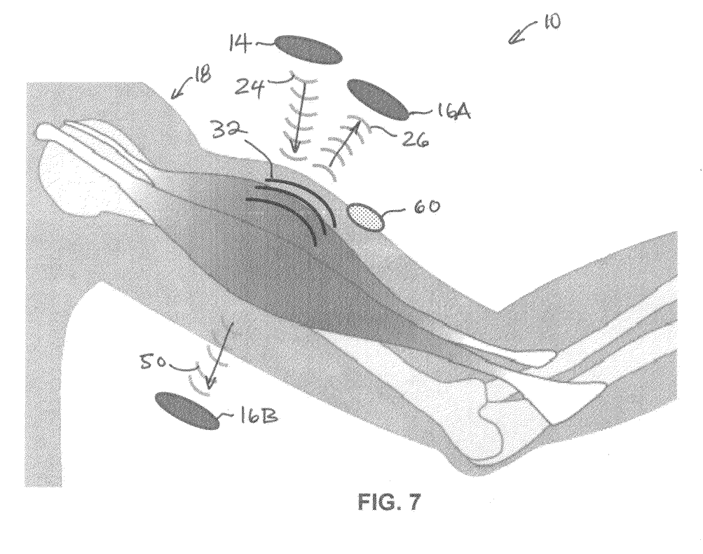

In FIG. 7, system 10 uses two types of resultant energy, namely reflected energy 26 and pass-through energy 50, to perform myography. Although a single transmitter 14 is illustrated, it will be appreciated that additional transmitters can be implemented.

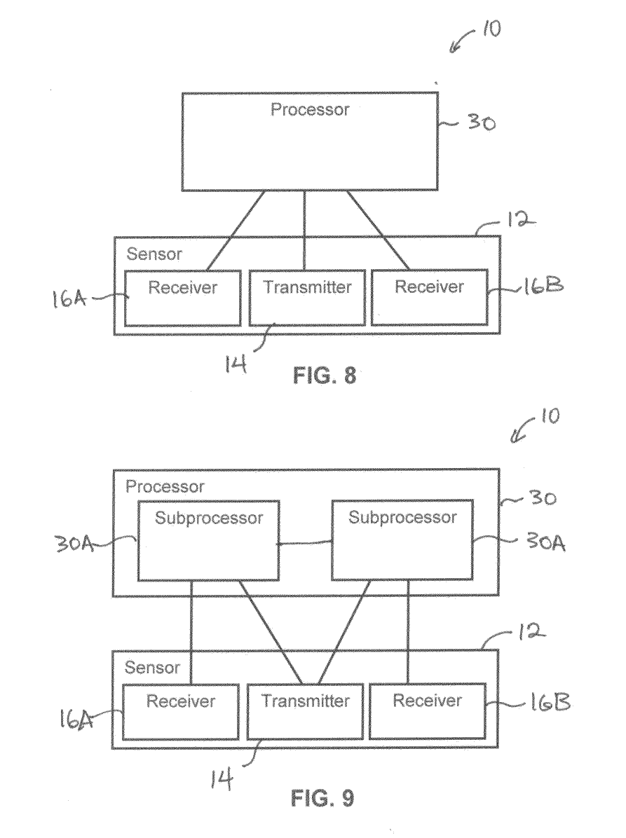

System 10 of FIG. 7 can be configured as shown in FIG. 8. In FIG. 8, receiver 16A is configured and positioned to detect reflected energy 26. Receiver 16B is configured and positioned to detect pass-through energy 50. Receiver 16A and receiver 16B are communicatively coupled to processor 30. Processor 30 is configured to analyze both reflected energy 26 and pass-through energy 50 detected by receivers 16A and 16B. For example, processor 30, receiver 16A and receiver 16B can be used to perform the methods of FIGS. 4 and 6 simultaneously or at separate times.

System 10 of FIG. 7 can be configured as shown in FIG. 9. In FIG. 9, receiver 16A is configured and positioned to detect reflected energy 26. Processor 30 includes subprocessor 30A and subprocessor 30B. Receiver 16A is communicatively coupled to subprocessor 30A. Subprocessor 30A is configured to analyze reflected energy 26 detected by receiver 16A. For example, subprocessor 30A and receiver 16A can be used in performing the method of FIG. 4.

Receiver 16B is configured and positioned to detect pass-through energy 50. Receiver 16B is communicatively coupled to subprocessor 30B. Subprocessor 30B is configured to analyze pass-through energy 50 detected by receiver 16B. For example, subprocessor 30B and receiver 16B can be used in performing the method of FIG. 6. Optionally, this can be done simultaneously and in coordination with performance of the method of FIG. 4 using subprocessor 30A and receiver 16A.

As used herein, the term “native signal” refers to an energy signal that originates from the muscle 20, such as electrical signals generated by muscle 20, audio signals generated by muscle 20, and vibration signals generated by muscle 20. Although reflected energy 26 and/or pass-through energy 50 may provide information on movements 32 associated with muscle vibration, it is to be understood that reflected energy 26 and pass-through energy originate from sensor 12 (transmitter 14 in particular) and is distinct from the native signals listed above. When determining muscle activity from reflected energy 26 and/or pass-through energy 50, processor 30 need not use data from any other sensor that might be present for the purpose of detecting a native signal. Processor 30, in determining muscle activity, can be configured to predict one or more physiological states through the use of resultant energy (e.g., reflected energy 26 and/or pass-through energy 50) without using native signals originating from muscle 20.

In further aspects, system 10 optionally comprises another sensor configured to detect one or more native signals originating from the body. Such a sensor can be referred to as native-signal sensor 60 to distinguish it from receivers 16 which sense resultant energy (e.g., reflected energy 24 and/or pass-through energy 50) that originates from transmitter 14. Although a single native-signal sensor 60 is illustrated in FIGS. 1, 5, and 7, it will be appreciated that multiple native-signal sensors can be used such as described in US Publication No. 2014/0163412.

Native signals can be any one or more of an electrical signal generated by muscle 20, an audio signal generated by muscle 20, and a vibration signal generated by muscle 20. For example and without limitation, each native-signal sensor 60 can be any of: (a) an electrode and associated electronics that can detect electrical signals generated by muscle 20, (b) a microphone (e.g., a piezoelectric device) that can detect audio signals generated by muscle 20, or (c) an accelerometer (e.g., a microelectromechanical device) that can detect vibrations generated by muscle 20.

Processor 30 can be communicatively coupled to each native-signal sensor 60. Processor 30 is configured to use the one or more native signals to determine the muscle activity. For example, the native signals can be used by processor 30 to derive a model of background noise in a manner described in US Publication No. 2014/0163412, and then the background noise can be subtracted from data obtained from receiver 16, 16A, and/or 16B. Native signals can also be combined with data obtained from receiver 16, 16A, and/or 16B, and then mechanomyography algorithms can be applied by processor 30 to the combined data to determine muscle activity. The process of combining may involve averaging, weighted averaging, and other techniques described in US Publication No. 2014/0163412.

Processor 30, in determining muscle activity, can be configured to predict one or more physiological states through the use of both resultant energy (e.g., reflected energy 26 and/or pass-through energy 50) originating from transmitter 14 and native signals originating from muscle 20. The predicted physiological state may include without limitation: (a) a degree to which the muscle is fatigued and (b) whether the muscle is contracting or not contracting.

In any of the above aspects, including those described in connection with any of the figures herein, processor 30 can be part of an electronic computer capable of executing, in accordance with a computer program stored on a non-transitory computer readable medium, any one or a combination of the steps and functions described. The non-transitory computer readable medium may comprise instructions for performing any one or a combination of the steps and functions described herein.

Processor 30 can be located at a distance away from body 18 and sensor 12, or processor 30 may be attached to body 18 or to sensor 12. Processor 30 can be a personal computer, such as a laptop computer or a desktop computer. Processor 30 can be an embedded digital device. As an embedded digital device, processor 30 can be embedded within one or more of sensors 16, 16A, 16B, or 60. Processor 30 can be a mobile digital device, such as a smart phone or tablet. Processor 30 can include subprocessors or components housed in separate devices, and such devices can be communicatively coupled to enable processor 30 to perform the steps and functions described herein.

While several particular forms of the invention have been illustrated and described, it will also be apparent that various modifications can be made without departing from the scope of the invention. It is also contemplated that various combinations or subcombinations of the specific features and aspects of the disclosed embodiments can be combined with or substituted for one another in order to form varying modes of the invention. Accordingly, it is not intended that the invention be limited, except as by the appended claims.

1. A myography method comprising:
transmitting energy toward a body having a muscle;
detecting resultant energy from the body, the resultant energy being any one or both of reflected energy and pass-through energy, the reflected energy including at least some of the transmitted energy that was reflected by the body, the pass-through energy including at least some of the transmitted energy that traversed across the body; and
determining muscle activity of the muscle using at least the resultant energy that was detected.

2. The method of claim 1, wherein the transmitted energy includes sound waves including any one or a combination of acoustic waves and ultrasonic waves.

3. The method of claim 1, wherein the transmitted energy includes radio waves.

4. The method of claim 1, wherein the transmitted energy includes infrared light or other frequencies of light.

5. The method of claim 1, wherein the resultant energy includes reflected energy that includes at least some of the transmitted energy that was reflected from any one or both of a skin surface and subcutaneous tissue of the body.

6. The method of claim 5, wherein the determining of muscle activity includes:
deriving muscle vibration data from the reflected energy, and
using the muscle vibration data in the determining of muscle activity.

7. The method of claim 6, wherein the deriving of muscle vibration data includes performing Doppler shift analysis of the reflected energy.

8. The method of claim 6, wherein the deriving of muscle vibration data includes performing frequency domain analysis of the reflected energy.

9. The method of claim 1, wherein the resultant energy includes pass-through energy that includes at least some of the transmitted energy that traversed across a skin surface and subcutaneous tissue of the body.

10. The method of claim 9, wherein the determining of muscle activity includes:
measuring, from the pass-through energy, a change in one or more muscle characteristics selected from the group consisting of energy reflectivity, energy absorption, and energy transmission; and
using the measured change in the one or more muscle characteristics in the determining of muscle activity.

11. The method of claim 1, further comprising detecting one or more native signals originating from the body, and wherein the determining of muscle activity includes using the one or more native signals in the determining of muscle activity.

12. The method of claim 11, wherein the one or more native signals are selected from the group consisting of an electrical signal generated by the muscle, an audio signal generated by the muscle, and a vibration signal generated by the muscle.

13. The method of claim 1, wherein the determining of muscle activity includes predicting one or more physiological states of the muscle.

14. The method of claim 13, wherein the predicting includes any one or both of (a) predicting a degree to which the muscle is fatigued and (b) predicting whether the muscle is contracting or not contracting.

15. A myography system comprising:
a transmitter configured to transmit energy toward a body having a muscle;
a receiver configured to detect resultant energy from the body, the resultant energy being any one or both of reflected energy and pass-through energy, the reflected energy including at least some of the transmitted energy that was reflected by the body, the pass-through energy including at least some of the transmitted energy that traversed across the body; and
a processor configured to determine muscle activity of the muscle using at least the resultant energy that was detected.

16. (canceled)

17. (canceled)

18. (canceled)

19. The system of claim 15, wherein the resultant energy includes reflected energy that includes at least some of the transmitted energy that was reflected from any one or both of a skin surface and subcutaneous tissue of the body.

20. (canceled)

21. (canceled)

22. (canceled)

23. The system of claim 15, wherein the resultant energy includes pass-through energy that includes at least some of the transmitted energy that traversed across a skin surface and subcutaneous tissue of the body.

24. (canceled)

25. (canceled)

26. (canceled)

27. (canceled)

28. (canceled)

29. A non-transitory computer readable medium having a stored computer program embodying instructions, which when executed by a computer, causes the computer to perform myography, the computer readable medium comprising:
instructions for transmitting energy toward a body having a muscle;
instructions for detecting resultant energy from the body, the resultant energy being any one or both of reflected energy and pass-through energy, the reflected energy including at least some of the transmitted energy that was reflected by the body, the pass-through energy including at least some of the transmitted energy that traversed across the body; and
instructions for determining muscle activity of the muscle using at least the resultant energy that was detected.

30. (canceled)

31. (canceled)

32. (canceled)

33. The non-transitory computer readable medium of claim 29, wherein the resultant energy includes reflected energy that includes at least some of the transmitted energy that was reflected from any one or both of a skin surface and subcutaneous tissue of the body.

34. (canceled)

35. (canceled)

36. (canceled)

37. The non-transitory computer readable medium of claim 29, wherein the resultant energy includes pass-through energy that includes at least some of the transmitted energy that traversed across a skin surface and subcutaneous tissue of the body.

38. (canceled)

39. (canceled)

40. (canceled)

41. (canceled)

42. (canceled)
